(19) United States
(12) Patent Application Publication
(10) Pub. No.: US 2025/0009928 A1
(43) Pub. Date: Jan. 9, 2025

(54) METHOD FOR ENVIRONMENTALLY-MODIFYING AIR WITHIN AN INDOOR SPACE

(71) Applicants: Kyler F. LUNMAN, II, Fernandina Beach, FL (US); Richard A. FERDINAND, Old Tappan, NJ (US)

(72) Inventors: Kyler F. LUNMAN, II, Fernandina Beach, FL (US); Richard A. FERDINAND, Old Tappan, NJ (US)

(21) Appl. No.: 18/711,597

(22) PCT Filed: Nov. 20, 2022
(86) PCT No.: PCT/US2022/080203

§ 371 Date: May 19, 2024

Related U.S. Application Data
(60) Provisional application No. 63/281,652, filed on Nov. 20, 2021.

(51) Int. Cl. A61L 9/20

(52) U.S. Cl. CPC A61L 9/20

(57) ABSTRACT

A method for environmentally-modifying air within an indoor space containing disease-causing pathogens by initially conveying a selected volume of air from the indoor space to an ultraviolet germicidal irradiation (UVGI) chamber. The selected volume of air is irradiated within the chamber while subjecting the selected volume of air to turbulence to obtain treated air in less than 2 seconds having at least a log 4 reduction of disease-causing pathogens. The treated air containing at least 99.99% inactivated pathogens is dispensed from the chamber to permeate back in to the indoor space to dilute and diffuse the disease-causing pathogens remaining in the indoor space. The irradiating step includes inactivating disease-causing pathogens in situ without contaminating or blurring the characteristics of the disease-causing pathogens so that the dispensed treated air consists of environmentally-modified breathable air with high acuity and sharpness of the inactivated pathogens.

### BACKGROUND OF THE INVENTION

### 1. Field of the Invention

The invention relates to a method for environmentally-modifying air within an indoor space.

### 2. The Prior Art

A With the occurrence of the Covid-19 pandemic, there is an increased interest in methods for disinfecting air that can be readily be placed into practice and have very high effectiveness exceeding 99%.

The present inventions are purposefully information rich combinations of the workings of vaccines, normally including aerosolized vaccines, that have been or may be administered to air-breathing creatures inclusive of members of the animal kingdom with an emphasis on humankind.

Prior tests of similar but less powerful in-class technology demonstrated green energy-saving optoelectronics-driven outcomes that are scalable to provide financially and socially beneficial environmental outcomes and incremental sources of funding for this new anti-pandemic environmental technology.

There are generally four main methods of UV air treatment. A first type comprises disinfecting an airstream via in-duct and/or air handling units in the HVAC system. A second type includes free standing units that recirculate air passed ultraviolet light (UV) lamps and fixtures. A third type includes Upper-Room Systems which treat air with multiple UV lamps hung from the ceiling or walls which are shrouded from the people below because humans cannot tolerate direct exposure to UVC. Finally, a fourth type comprises Barrier Systems which are normally hung in the overhead portion of the door with louvers to constrain the UVC rays.

However, these methods of UV air treatment have drawbacks which limit their application. Recirculation units are comparatively smaller and normally sit in a corner of a room or area thereby treating very small areas. Upper room and Barrier types normally have no ability to control or direct airflow either to or away from their unit. In many of these systems the areas have to be evacuated for them to be utilized. Additionally, these UV air treatment types are not scalable so they are unable to irradiate airborne and surface pathogens in different sized areas or airspaces.

One approach is detailed in our prior U.S. Pat. No. 9,675,726 entitled Scalable Airborne Pathogen Removal System which describes an air handling system where circulated air is exposed to ultraviolet light. However, recent discoveries in immunology and social health management require a more robust system with increased effectiveness.

Therefore, a need exists for novel methods configured for the irradiation of airborne and surface pathogens using ultraviolet light. There also exists a need for novel ultraviolet germicidal irradiation (UVGI) airborne pathogen removal air methods which can be employed anywhere. Finally, there exists a need for scalable ultraviolet germicidal irradiation methods for airborne pathogen removal.

Furthermore, implementation of the present invention, with or without the prior known air-handling methods, may include a multiplicity of new methods, sequences and cycles that disable, kill, eradicate and/or interdict (slow, deter or stop) formations of infectious concentrations of disease caused by viruses, bacteria and fungi, along with many additional pollutants and contaminants.

### SUMMARY OF THE INVENTION

These and other related objects are achieved according to an embodiment of the invention by a method for environmentally-modifying air within an indoor space containing disease-causing pathogens. The method initially includes conveying a selected volume of air from the indoor space to a UVGI chamber. Then irradiating the selected volume of air within the chamber while subjecting the selected volume of air to turbulence at a Reynold's Number (Re) between about 4,000 and about 5,000 to obtain treated air in less than 2 seconds having at least a log 4 reduction of disease-causing pathogens. Next dispensing the treated air containing at least 99.99% inactivated pathogens from the chamber to permeate back in to the indoor space to dilute and diffuse the disease-causing pathogens remaining in the indoor space. And subsequently inactivating disease-causing pathogens in situ during said irradiating step without contaminating or blurring the characteristics of the disease-causing pathogens so that the dispensed treated air consists of environmentally-modified breathable air with high acuity and sharpness of the inactivated pathogens.

A further step includes recirculating the diluted and diffused disease-causing pathogens through the UVGI chamber for homogenizing all air within the indoor space to at least a log 4 reduction of disease-causing pathogens separate and independent from an HVAC system servicing the indoor space. The dispensing step comprises dispensing the treated air from the chamber to permeate back in to the indoor space for diluting and diffusing the disease-causing pathogens remaining in the indoor space to reduce a viral load and a bacterial load of the disease-causing pathogens.

The irradiating step includes emitting ultraviolet radiation between 20-35 kWatts/m2 within the UVGI chamber; emitting ultraviolet radiation in the range of 250 and 280 nm, inclusive; and retaining the selected volume of air within the chamber for a dwell time of about a 1 second. The combination of features in the irradiating step provide at least a log 5 reduction of disease-causing pathogens.

The inactivating step further includes preserving a biological characteristic of a disease-causing bacterial pathogen and preserving a genomic characteristic of a disease causing viral pathogen so that the biological characteristics are present in the deactivated pathogens. The preserved biological characteristics of the inactivated pathogens are adapted to safely trigger an immune response in an occupant of the indoor space without risk of infection. The preserving step includes preserving the morphology, antigenic properties and immunogenic properties of disease-causing viral pathogens whereby the inactivated viral pathogens are adapted to induce production of virus-neutralizing antibodies in mammals present within the indoor space without risk of acquiring infectious disease.

The retaining step includes subjecting the selected volume of air to the Coanda Effect so that a portion of the selected volume of air hugs an interior surface of the UVGI chamber to increase dwell time. The conveying and dispensing step includes advancing the selected volume of air through passageways of varying widths to induce pressure and velocity differentials via the Bernoulli Principle to increase a throughput of treated air back in to the indoor space. Subjecting the selected volume of air to turbulence in combination with subjecting the selected volume of air to the Coanda Effect and advancing the selected volume of air through passageways of varying widths induces pressure and velocity differentials to provide sufficient irradiance dosage to dispense at least 100 cubic feet of treated air per minute with a log 6 reduction of disease-causing pathogens. The dispensing step consists of dispensing the treated air containing at least 99.99999% deactivated pathogens from the chamber to permeate back in to the indoor space to dilute and diffuse the disease-causing pathogens remaining in the indoor space so that the entire indoor space contains antiseptic breathable air.

The irradiating and retaining steps provide effective levels of inactivating dwell time, wherein the conveying, dispensing and advancing steps provide throughput, and wherein the method further includes the step of balancing the effective levels of inactivating dwell time against throughput for obtaining a target irradiance as a product of flux, distance, time and UVGI wattage.

### BRIEF DESCRIPTION OF THE DRAWING

The advantages, nature, and various additional features of the invention will appear more fully upon consideration of the illustrative embodiments now to be described in detail in connection with accompanying drawings. In the drawings wherein like reference numerals denote similar components throughout the views:

FIG. 1 is a flowchart presents a summary of the steps in the method according to the invention.

FIG. 2 is another flowchart illustrating certain steps included within the irradiating and inactivating processes.

FIG. 3 is a further flowchart presenting various steps that are part of the conveying, dispensing and recirculating processes.

### DETAILED DESCRIPTION OF THE PREFERRED EMBODIMENTS

The inventors have invented and engineered the initiating systems, methods and processes of the present inventions to combat spreading of communicable diseases that may become pandemic diseases via interdiction which is an environmental mode of operation that distorts and disrupts conditions outside of the human body making said conditions incompatible with normal avenues of airborne infectivity and transmissibility that enable protected indoor instances of viruses, bacteria and fungi to spread aggressively enough to lead to pandemics. The inventors have also engineered the initiating systems, methods and processes of the present inventions to enable economical mass production of antiseptic breathable air.

Throughout this Specification the various definitions will be provided. In the context of the present inventions, a vaccine is a biological substance and/or a genomic information set and/or, in the unique context of the leading present invention detailed herein, a sequence of physical steps in the production and circulation of antiseptic breathable air, that, in accordance with this invention, are necessary precursor processes integrated into the production of specially pure and acutely defined antigens that are newly originated to become feedstocks of human immune systems and to act as as related vehicles of biological/genomic instructions wherein said physical steps in the production and circulation of antiseptic breathable air may automatically and immediately be used to stimulate the initial production of newly originated antigens and subsequent production and usage of related antibodies by human or zoonotic immune systems and, as a continuum, simultaneously or sequentially, provide immunities against one or many airborne agents of infectious pathogens and allergens. In accordance with the present invention(s), multiple cases and sequences of complex cross immunizations of varying consequences are feasible to be created within brief exposure periods. These may effect persons or other air-breathing creatures in the immunization sending or receiving groups. Achievements, recordings and resultant understandings of these complex cross immunizations are uniquely delivered, highly valuable, results of the exercise of the present invention(s). The benefits of achieving natural immunity are described in the Research Papers: Gazit, et al, Comparing SARS CoV-2 Natural Immunity to Vaccine-Induced Immunity: Reinfections Versus Breakthrough Infections, 2021; and Shrestha et al, Necessity of COVID-19 Vaccination in Previously Infected Individuals, 2021, the contents of which are incorporated herein by reference thereto.

The uniformity of fullness, speed and acuity of understandings of these potential complex cross immunizations are unique protective advantages derived from and dependent upon utilization of antiseptic air conditions during the precursor processes stipulated in these present inventions.

Explanation: “are feasible within brief exposure periods. Explanation: The uniqueness of the present invention is accentuated by the inclusion of the verbiage: a continuum of physical process steps in the manner in which the cause and effect of immunization itself is executed, defined and described herein. Usage of this special linguistic expression is generated and justified by intrinsic systemic process steps such a “diffusion”, “dispersion”, “permeation” and “homogenization” which serve to interdict infectivity and transmissibility of disease as intrinsic effects of the complex originating inoculation process itself.

The process of interdiction is why the inventors refer to the immunization processes of the present invention as “environmental”. In fact, during an exemplary time frame of operation of the present invention, the entirety of infectious pathogenic and allergenic potentials within a protected space may be “inhaled” by the systems of the present invention into the physical optoelectronic channels and chambers of the Invention wherein infectivity and transmissibility of disease vectors are repeatedly disabled and populations of pathogens and allergens may, outside of the human body in some cases, be subject reductions of 6 log (1,000,000 to 1), or greater, which is generally accepted as antiseptic. These “interdictive” process steps are not defined herein as medical because, in addition to producing medical outcomes, they may occur and may effect conditions, circumstances and outcomes unrelated to medical conditions of air breathing creatures.

Log reduction is a measure of how thoroughly a decontamination process reduces the concentration of contaminant. It is defined as the common logarithm of the ration of the levels of contamination before and after the process, so an increment of 1 corresponds to a reduction in concentration by a factor of 10. In general, an //-log reduction means that the concentration of remaining contaminants is only 10-// times that of the original. So for example, a 0-log reduction is no reduction at all, while a 1-log reduction corresponds to a reduction of 90 percent from the original concentration, and a 2-log reduction corresponds to a reduction of 99 percent from the original concentration.

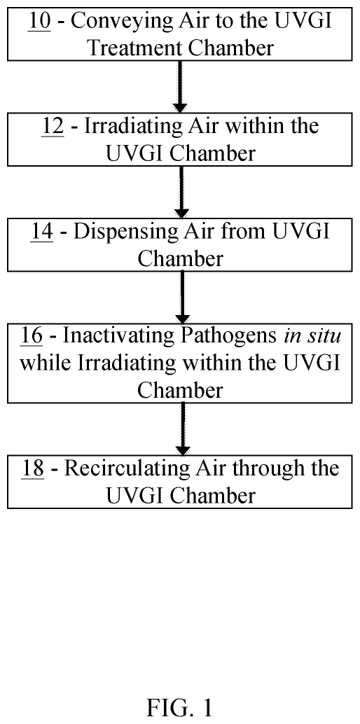

Referring now in detail the flowchart of FIG. 1, there is illustrated an overview of the method for environmentally-modifying air within an indoor space containing disease-causing pathogens. Initially the method includes conveying 10 a selected volume of air from the indoor space to a UVGI chamber. The selected volume of air is then irradiated 12 within the chamber. The treated air containing at least 99.99% deactivated pathogens is then dispensed 14 from the chamber back in to the indoor space. During the irradiating step 12, disease-causing pathogens are inactivated in situ within the UVGI chamber. Since the method operates continuously, air is recirculated 18 through the UVGI chamber until the entire indoor space has at least a log 4 reduction of pathogens, equivalent to the levels in the dispensed air. The recirculating step 18 operates separate and independent from an HVAC system servicing the indoor space.

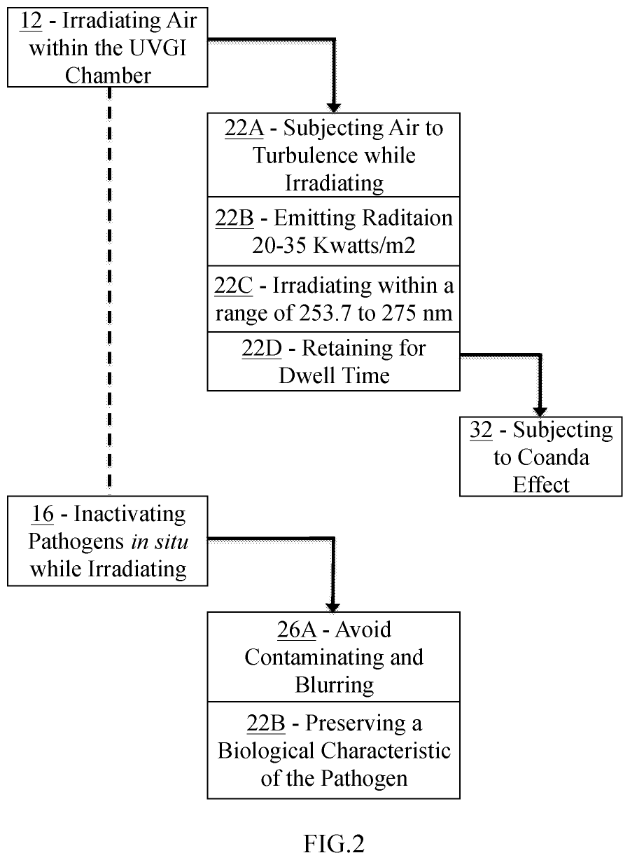

As can be seen in the flowchart of FIG. 2, irradiating 12 includes multiple embedded steps. These include subjecting 22A the selected volume of air to turbulence at a Reynold's Number (Re) between about 4,000 and about 5,000 to obtain treated air in less than 2 seconds having at least a log 4 reduction of disease-causing pathogens. In addition, irradiating is characterized by emitting 22B radiation between about 20-35 Kwatts/m2 within an irradiating 22C range of 253.7 to 275 nm, inclusive. Proper treatment requires retaining 22D all volumes of air within operative distance of the UVGI source for a minimum dwell time or residence time. One technique to increase dwell time is subjecting the conveyed air to the Coanda Effect, in which airflows hug an interior surface of the UVGI chamber near the UVGI source or in a direction toward the UVGI source. While inactivating 16, the method is carefully tuned to avoid contaminating and blurring 26A the characteristics of the disease-causing pathogens. This type of pure inactivating 16 results in preserving 22B a biological characteristic of the pathogen.

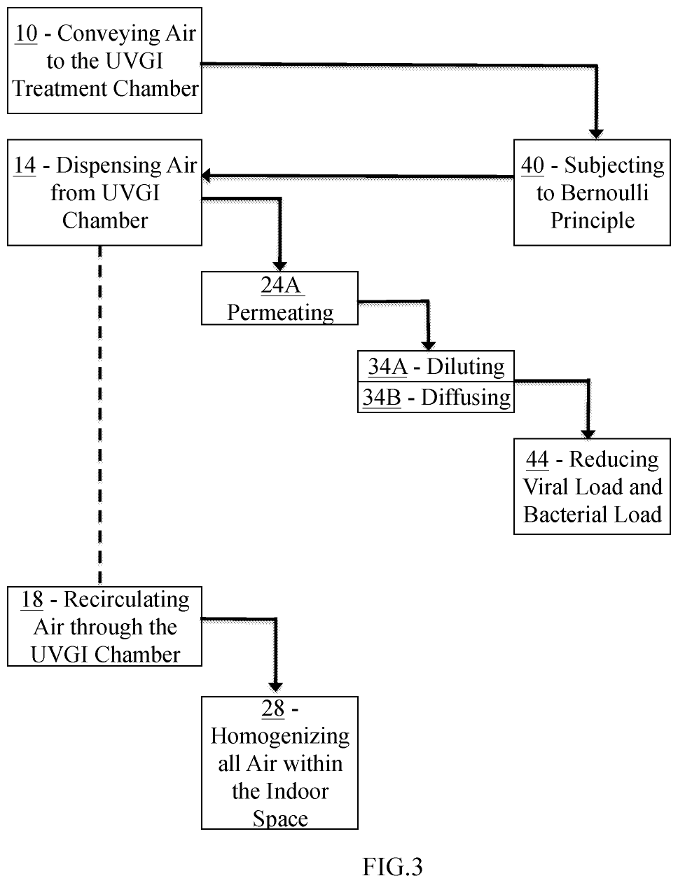

Additional step are illustrated in FIG. 3, where the treated air containing at least 99.99% deactivated pathogens is dispensed 14 from the chamber for permeating 24A back in to the indoor space for diluting 34A and diffusing 34B the disease-causing pathogens remaining in the indoor space. During the irradiating step, disease-causing pathogens are inactivated 26 in situ without contaminating or blurring the characteristics of the disease-causing pathogens so that the dispensed treated air consists of environmentally-modified breathable air with high acuity and sharpness of the deactivated pathogens. The diluted and diffused disease-causing pathogens are then recirculated 18 through the UVGI chamber for homogenizing 28 all air within the indoor space to at least a log 4 reduction of disease-causing pathogens separate and independent from an HVAC system servicing the indoor space.

The UVGI chamber functions as a ducted treatment housing and also as a protective enclosure for containing the UV rays. There is at least one input duct through which air is conveyed from the indoor space into the chamber. There is at least one output duct through which air is dispensed from the chamber back to the indoor space. The interior of the chamber is equipped with reflective surfaces to direct UV rays back into the chamber. The surfaces are also contoured to induce turbulence to mix the conveyed air. The contoured surfaces also direct conveyed air into close proximity to the UV radiation source and insure all portions of the conveyed air will experience sufficient dwell time. The combination of mixing the air and providing sufficient dwell time insures that all irradiated air has at least a log 4 reduction in disease-causing pathogens.

Another aspect illustrated in FIG. 3 relates to the volume of air processable per unit time. In laboratory settings, it is possible to tightly control distance and time for exposing disease-causing pathogens to UVGI radiation. However inactivating pathogens in situ for large spaces requires fast processing while maintaining sufficient irradiance. Irradiance is the dosage calculation of flux, distance, time and UVGI wattage which is calculated internally to deliver the highest possible UVGI dosage in wattage/m2 to inactivate (breaking the covalent bond) the DNA of microorganisms. In addition to irradiance, there are 12 other variables of air disinfection. The method converts laboratory levels of disinfection and scales them to commercial production levels. Accordingly, all of the above variables are balanced, calculated individually and simultaneously compared collectively to develop the highest possible dosage to inactivate the DNA of any microorganism. By way of example, having air hug the interior walls under the Coanda Effect allows control of airflow near or towards the UVGI source. In combination therewith, the method conveys 10 the same airflows to passageways of varying widths before dispensing 14 into the room. This modifies pressure and velocity within the chamber thus subjecting 40 the airflows to the Bernoulli Principle. Thus, airflow hugs surface to exceed minimum dwell time, is mixed (turbulence) to insure complete inactivation and is accelerated to maintain high flow rates. This combination of inducing turbulence and subjecting to the Coanda Effect 32 and the Bernoulli Principle 40 are key aspects of the method.

Thus, the treated air has at least 99.99% deactivated pathogens compared to the air initially conveyed into the UVGI treatment chamber. The treated air is dispensed from the UVGI chamber to permeate back in to the indoor space. In this context, permeate means that the treated air will pass through every part of the indoor space. When permeating, the treated air will dilute and diffuse the disease-causing pathogens remaining in the indoor space. Dilute means to reduce the overall concentration of disease-causing pathogens within the indoor space. Diffuse means the remaining diseasecausing pathogens will be spread out throughout the indoor space. By diluting and diffusing the disease-causing pathogens, occupants of the indoor space will be exposed to a lower bacterial, fungal or viral load. A lower viral load, for example, affords the immune system time and opportunity to develop immunity to the virus without causing illness. Smaller prior art disinfection systems or residential disinfection units can only treat small volumes of air. This can result in pockets or layers of disinfected air in parts of a room, while disease-causing pathogens can remain in high concentrations in other parts of the room. The method according to the invention overcomes this limitation by conveying and dispensing large volumes of treated air. This is achieved by having a high flow rate through the UVGI treatment chamber. The high flow rate, or high throughput, will be discussed in greater detail below in connection with the control surfaces.

Dispensing treated air to permeate back in to the indoor space to dilute and diffuse the disease-causing pathogens has numerous benefits. Some of those benefits are documented in a scientific medical report: Immunogenic Properties of SARS-CoV-2 Inactivated by Ultraviolet Light, Gracheva et al, Archives of Virology (2022) 167: 2181-2191 published on line on Jul. 20, 2022, the contents of which are incorporated herein by reference thereto. The method provides for inactivating disease-causing pathogens in situ during said irradiating step to maximize those benefits. In this application, in situ means the inactivating occurs in the original place, that is within the indoor space or immediately adjacent the indoor space. The actual location of the UVGI chamber could be mounted on the ceiling or within the ceiling with ducts through the ceiling into fluid communication with the indoor space. The inactivating is also occurring immediately and continuously.

Traditionally, attenuating or inactivating a pathogen occurs under laboratory conditions in a process taking several days. Such processes are neither in the original location of the virus, nor immediate nor continuous. As indicated in the Gracheve report, “Widely used chemical methods of virus inactivation involve the use of highly toxic substances such as B-propiolactone, classified as a potent carcinogen to humans (group 2B carcinogen), and formaldehyde, classified as carcinogenic to humans (group 1 carcinogen) by the International Agency for Research on Cancer [41], For this reason, physical methods of virus inactivation such as UV irradiation are preferred in vaccine production technology, as they do not involve the treatment of the virus with harmful substances and therefore do not require the introduction of additional steps to purify the viral antigen from toxic compounds.” The method according to the invention inactivates disease-causing pathogens during the irradiating step without contaminating or blurring the characteristics thereof. In the context of the invention, the term contaminating has is usual meaning, that being free from foreign substances, or pure. Similarly, “blurring” of the eventual stored images, data or data sets is constrained because of deliberate antiseptic controls, limiting of contaminants, limiting of manufacturing residues, and unambiguous emphasis on overall start to finish process speeds in the interest of constraining types biological decay that require process time that can be reduced.

As summarized in the Gracheve report, “This study demonstrates that the UV treatment of SARS-CoV-2 completely inactivates its infectivity while preserving its morphology, antigenic properties, and ability to induce production of virus-neutralizing antibodies in mice immunization.” Gracheve comments that, “The development of whole-virion inactivated vaccines is of particular interest, since such vaccines include the full set of structural viral proteins. The assurance of complete inactivation of the virus coupled with retaining the native conformation of the protective antigens is one of the most important requirements for whole-virion vaccines. Finally, Gracheve states, “[T]he effectiveness of physical methods of virus inactivation, such as ultraviolet irradiation of the virus stock, remains relevant. The aim of this work was to evaluate the effect of the SARS-CoV-2 virus inactivation with ultraviolet light (UV) on its morphology and antigenic and immunogenic properties.”

The inventive method preserves virus morphology and antigenic and immunogenic properties by dispensing air that consists of environmentally-breathable air with high acuity and sharpness of the deactivated pathogens. In the context of the present invention, “acuity” means “sharpness of the antigen image and/or precision and completeness of the garnered data in any form or format, such that the human immune systems can recognize and/or readily identify the virus morphology and antigenic and immunogenic properties. Morphology refers to the size, shape and structure of microorganisms and the relationship between their constituent parts.

The diluted and diffused air is then continuously recirculated through the UVGI treatment chamber. The dispensing and recirculating within a short period of time will provide for homogenizing all air within the indoor space to at least a log 4 reduction of disease-causing pathogens. Within the context of the invention, homogenizing means mixing until a consistent concentration of disease-causing pathogens is present throughout the entire indoor space. Shortly after the method is commenced, the recirculating and homogenizing functions will reduce the disease-causing pathogen level to the same level as the dispensed treated air, that is, to an disease-causing pathogen level less than 0.01%.

As the processed breathable air that is non-infectious or even antiseptic is circulated throughout the breathable air within the protected space, it is repeatedly dispersed by fan supplied energy, or other air moving technologies, to the effect that infectious concentrations, if any, are also dispersed, thus reducing infectivity and transmissibility in connection with each repeating dispersal and said breathable air is further dispersed and diffused by the natural tendency for gases to expand. In addition, some volume of these circulating airborne pathogens and allergens within the circulating breathable air, is repeatedly “inhaled” by the originating physical systems into the physical optoelectronic channels and chambers of the invention wherein infectivity and transmissibility of airborne disease vectors are again repeatedly disabled and populations of pathogens and allergens may, in some cases, again be subject to optoelectronic ultraviolet germicidal irradiation (UVGI) reductions of 6 log (1,000,000 to 1) or greater which is generally accepted as antiseptic.

The present invention may use various known or new air-handling methods, processes and sub systems that, when used to implement the present invention in novel ways, via application of ultraviolet germicidal irradiation (UVGI) and/or other forms of purposed energy emissions potentially including narrow and multi spectral light emissions; e-beams; avalanche dump laser light emissions, x rays, LED's and ultra sound; among others to achieve a sequence of physical events that, in whole or in part may interdict (slow, deter or stop), disable and/or kill airborne biological agents of potential infections, contagions and pandemics.

An air flow management system is located between the inner boundary walls and the source of ultraviolet irradiance to provide an exposure slot having a cross-sectional area receiving at least 20 Kwatts/m2. An air motivator, like a fan or blower, is configured to draw air from the airspace into the air intake through the treatment chamber in a downstream direction and expel treated air out of the air output back to the airspace. The cross-sectional area of the exposure slot is configured and dimensioned to provide all drawn air with at least 360 milliseconds of dwell time within the exposure slot to produce treated air with at least 99.99% of pathogens eradicated while maintaining a throughput of about 120 units of air per minute drawn into and expelled out of the pathogen removal system.

The source of ultraviolet irradiance between 20-35 kWatts/m2 across the entirety of the exposure slot. The source of ultraviolet irradiance is selected from a longitudinally-extending ultraviolet lamp, bulb, or LED that generates oriented parallel to the terminal end of the gate section through 95-100% of the treatment chamber emitting irradiance in the range of 250 to 280 nm, more particularly between 253.7 and 275 nm, inclusive.

The air flow management system directs air drawn in to the treatment chamber through one or more stages serially-aligned in the downstream direction. Each stage includes a symmetrically increasing cross-sectional area that extends past the source of ultraviolet irradiance followed by a symmetrical reduction in cross-sectional area of 30-70%. A contour profile of the continuous ribbon in conjunction with a filtered volume of air drawn in to the treatment chamber mechanically balances induced turbulence for extended dwell time with throughput as a function of unit size while the location and reflectivity of the continuous ribbon in conjunction with a power output and configuration of the UV source photooptically maximize the irradiance of all particles. Air entering the ramp section hugs the continuous ribbon according to the Coanda Effect to increase dwell time. A discontinuity in the ribbon increases the turbulence of the air flow at a side wall of the treatment chamber; wherein the continuous ribbon induces turbulence characterized by a high Reynolds number, preferably an Re of between 4,000 and 5,000.

The Reynold's Number (Re) is calculated by the following formula.

$${Re}_{D} = \frac{ρ VD}{μ} = \frac{VD}{v}$$

An Re less than 2000 is considered low velocity, fluid motion generally in a straight line with virtually no mixing between layers. In contrast, an Re greater than 4,000 is considered high velocity, in which particles within the fluid experience irregular motion. The combination of airflow hugging the walls, and high turbulence allows thorough inactivating while maintaining a high throughput volume of air.

The present method provide inactivated pathogens which function as vaccines since they are derived from the causative agents of the underlying known or unknown affliction in-situ, at high speeds and with exceptional end product acuity, i.e. antigen and antibody acuity. As concluded by Gracheva et al, “Thus, the UV inactivation of SARS-CoV-2 makes it possible to obtain viral material similar in its antigenic and immunogenic properties to the native antigen, which can be both for the development of diagnostic test systems and for the development of an inactivated vaccine against COVID-19.” These vaccines-type by products have important competitive advantages versus conventional alternatives because of the following.
(A) The present inventions incorporate an environmental process effect that, within the pathogen to antigen conversion processes of the present inventions, reduces airborne concentrations of infectious agents by at least 6 log (a pathogen reduction of one million to one), a level that is generally regarded to be antiseptic as later defined herein
(B) Furthermore, the present invention is a continuum wherein disabling of infectious airborne pathogenic agents by photonic bombardment occurs when the system-inhaled air that has been exhaled by humans, or other air breathing creatures, is exposed, within a safe optical irradiation chamber (or chambers) to sufficient dosages of ultraviolet germicidal irradiation (UVGI), occasionally in conjunction with other optical frequencies not discussed herein, resulting in the conversion of known or unknown airborne pathogen(s) into antigens including zoonotic conversions.
(C) In order to gamer and collect human benefits during and after the period of optical irradiation, it is a unique attribute of the present invention that, when circumstances of the particular instance of utilization of the present invention demand the highest purity, acuity and quality of antigens, the antigens produced by the present Invention may be superior for subsequent examination, recognition identification, categorization, labeling and short and long term storage by the human or animal immune systems as actors; and/or, by human analysts and their information devices as students, teachers beneficiaries and tool users in any capacities.

In the context of the present invention, “acuity” means “sharpness of the antigen image and/or precision and completeness of the garnered data in any form or format, such that the human immune systems can recognize and/or readily identify a single antigen among many antigens. It is very relevant that “blurring” of the eventual stored images, data or data sets is constrained because of deliberate antiseptic controls, limiting of contaminants, limiting of manufacturing residues, and unambiguous emphasis on overall start to finish process speeds in the interest of constraining types biological decay that require process time that can be reduced. The sense of this feature of the present invention is that overall process speed can improve overall data quality to the benefit of immunization and that the systems, methods and processes of the present invention executed in-situ among humans and/or non human air breathing creatures during periods when they may in-situ inhale or exhale at least some of the same breathable air may be managed to become a benefit to virtually all air breathing creatures.

A combination or combinations of work products of the present invention, may be deliberately assembled in data sequences that produce accumulating semantic richness for diagnosticians and researchers that is further combined with knowledge of intermittent artificially produced 6-log or better antiseptic breathable air that is available throughout the systems of the invention as an in-situ disinfectant, transmissibility diluting medium, infectivity diffuser and, importantly, as an in-situ infectivity reference and quality control standard. As an indicator of the relevance of this reference and control standard, a process manager might want to pay particular attention to irradiation chambers' light energy indicators when a new infectious pathogenic variant is suspected or discovered in his or her locale. In this context, the relevant system may serve as a first instance indicator of the presence of a new infectious pathogenic variant in the relevant locale. First instance recognition may save thousand of lives. In this case an extra maintenance checkup generated by an active “maintenance-needed” optical signal could deliver exceptionally valuable human benefits.

The “first instance” recognition potential of the present method is described in the following context. When considering a volume of circulating air that is being, or has already been, disinfected to one or more 6 log reductions of biological pathogens and allergens via photonic bombardment, then the antiseptic air itself, for differing process reasons, becomes a disinfectant because it dilutes and also, as gas, naturally diffuses adjacent air by expansion. It is widely accepted that increasing the concentrations of airborne pathogens and allergens increases their infectivity and transmissibility, therefore the opposite is true and, in this context, dilution and diffusion are disinfectants and are, as contextual features of the present invention, beneficial characteristics of breathable air that has been disinfected to one or more 6 log reductions. The importance of these features increases with the perceived danger of the interdicted pathogens and/or allergens.

Operating together, these features of the present invention introduce fast new diagnostically directional healthcare information engagement platforms and systems that are deliberately imbued with sequences of relevant: recognition signals; identification signals; and process transition markers.

Process transition markers, in the context of the present invention, are indicators or instructions such that, at the marker point, the processes of the present invention will divert data in any selected form to flow toward an ancillary set of one or more additional chambers, including potential additional waste chambers or optical irradiation chambers, because different subsequent types of work processes are planned or anticipated. Identification of said outcomes are not within the purview of the present invention.

Thus, one of the goals of the present invention is to provide researchers with data that feeds accumulating intuitions, developing mental pictures and other hints that are best assembled and reassembled by the human mind. This feature of deliberate sequential thought provocation to the benefit of further invention and understanding by end users is regarded as a legitimate claim of the invention because some of the intelligence and data targets of the present invention are mutable and everchanging. Humans may naturally analyze and think ahead in ways that are not computer programmable or otherwise subject to predictive automation. Therefore the present invention provides unique and novel benefits to human end users.

In the real world, dependable antiseptic air environments are required in order to optimally execute the systems, methods and processes of the present invention such that, as a result, said systems, methods and processes will be better able to work together with the necessary speed and acuity. When speed and acuity are not adequate, the protective effectiveness of the human and other targeted immune systems may degrade accordingly.

Although the present Invention has built-in repeating and compensating antiseptic performance buffers, it is best to keep all contributing factors toward antiseptic status in full operating mode at all times. For example, multiple sequential passes of circulating air through 6 log reductions tends to be a constant powerful reinforcement of process completions. The inventors recommend routine inspections and spot tests aimed at keeping all contributing factors toward antiseptic status in full operating mode at all times.

The extended scope of the present invention: also includes patent protected varieties of automated data exchanges utilizing recognition among administered vaccines of all kinds, artificially produced antiseptic breathable air and the workings of human and other immune systems as described herein and offered as exemplary of the information engagement and beneficial individual and public health effects thereof on all air-breathing creatures inclusive, for example, of cross referencing and cross tracking disease vectors separately among human populations of any size and among populations of non human air breathing zoonotic creatures of any type or size.

In the context of the present invention, the expression “antiseptic air” means breathable air in which airborne infectious pathogens have been disinfected by multiple safe exposures to safely applied ultraviolet germicidal irradiation such that at each instance of exposures an antiseptic disinfection capacity or event occurs that reduces the number of infectious agents in the irradiated air stream to one in a million or less (a six log reduction) which is a level that is generally accepted as antiseptic. Antiseptic is defined as “relating to or denoting substances that prevent the growth of disease-causing microorganisms.”

The inventors have engineered the method of the present inventions to combat spreading of communicable diseases that may become pandemic diseases via inactivation or interdiction which is an environmental mode of operation that distorts and disrupts conditions outside of the human body making said conditions incompatible with normal avenues of airborne infectivity and transmissibility that enable protected indoor instances of viruses, bacteria and fungi to spread aggressively enough to lead to pandemics.

The inventors have also designed the methods to enable economical mass production of antiseptic breathable air. This provides reliable maintenance of non-infectious status of breathable air within protected indoor spaces coupled with scalability to the upper limits of permitted human occupancy in accordance with applicable fire codes.
(I) As a beginning benchmark, the inventors recommend that initial failsafe provisioning for every protected space should be designed to protect, as an example, a 1,000 square feet room with a ten feet ceiling occupied by 67 large military males standing in lines and occupying 15 square feet per large male.
(II) At this maximum permitted occupancy, each space would be approximately 3 feet by 5 feet which is a very tight space that is chosen from public records to describe the worst case that the disinfection technology might need to overcome.
(III) With each large male (of 67) exhaling 14 cubic feet foot per hour there will be 938 cubic feet of exhalations per hour processed within the ultraviolet germicidal irradiation chamber (IV) The invention's 6 Log antiseptic systems run constantly, disabling biological airborne pathogens instantaneously at some point during 2 second homogenized airstream exposures
(V) One embodiment of the disinfection method processes approximately 2,000 cubic feet per hour.
(VI) The frequency of 6 Log or better exposures is higher than 2,000 cubic feet per hour.

The 6 log or better antiseptic air constantly recirculating in spaces protected by the workings of the present invention is permeated with recently created antigens that purposefully exhibit exceptional uniformity and acuity enabled by antiseptic conditions with respect to reliable and repeatable characteristics that can be “recognized” as same or similar by human immune systems that are executing high speed exploratory recognition surveys of new and pre existing antigens that may be available for decades as improvements to the protective response performance of human immune systems.

The present invention creates a novel loop feedback opportunity for analysts wherein specific process related sequences of pathogens to antigens to antibodies may be uniquely recognized, identified and categorized by human examiners and their devices with the end goals of better understanding the relationships in human friendly terms.

The data regarding antigens discovered by the human immune system while conducting the high speed “recognition” surveys are characterized and selectively stored for future information value to T cells or B cells. The exceptional uniformity of the captured data resulting from antiseptic air environments makes the immune systems' searchable “recognition” characterization processes faster, more accurate and more repeatable. Thus, the Present Invention enables the human immune systems to protect their human hosts in a faster, more accurate, more durable and more repeatable fashion.

The results of the searchable “recognition” characterization processes are then studied by the immune systems' slower, more precise “identification” characterization processes. This step is comparable to when diagnosticians make final choices of a specific prescription after many options have been considered.

The methods described herein are executed as a continuum that is actually a schema of scientific reasoning that nature embeds at some level in all of its living creatures. Human immune systems appear to have evolved this schema at an extraordinary rate over the last century of modern technology and extreme human mobility.

The methods of Optoelectronic Continuous Disinfection Devices, as depicted herein, operating as individual units designed to constantly maintain disinfected non infectious and/or antiseptic air circulation that diffuses irradiated and photon bombarded antiseptic breathable air within hospitals, traditional offices, indoor spaces or enclosed spaces also including, among others, tents and vehicles as examples wherein the antiseptic quality of the antigen rich or antibody rich air samples makes said samples safer to handle, ship and characterize.

The diffused irradiated antiseptic breathable air is thereafter permeated with newly created antigens that will in some cases, and may in most cases, contain detectable, recognizable and identifiable snippets of nucleic acids wherein controlled creation of said snippets of antigens within antiseptic air environments results in snippets that are generally more uniform than snippets produced by any known alternative method of large scale in situ antigen generation

Unique levels of uniformity for purposes of antigen characterization via snippets which, thereafter, uniquely enables more rapid and accurate recognition and/or identification of sampled antigens

Type 1 recognition characterization scanners of human immune systems will be high volume systems that will rapidly tag and report certain of said snippets as recognizable individual characterization instances in accordance with proprietary characterization reporting templates that will record and report certain recognizable characteristics that will thereafter be used to describe snippet-based linkages among relevant detected pathogens, allergens, antigens and antibodies. The methods described herein utilize recognition and identification of snippets of nucleic acids

The second type of characterization scanners of human immune systems will be type 2 lower volume/higher speed, more precise and selective, characterization systems that will tag certain of said snippets as identifiable nucleic acid sample instances that are common to two or more samples of nucleic acids regardless of whether said samples are DNA or RNA snippets of pathogens, allergens, antigens or antibodies.

Thus, the production and utilization of unique type 2 low volume characteristic scanner systems that will rapidly tag and report certain of said snippets as identifiable individual characterization instances in accordance with proprietary characterization reporting templates that will record and report certain recognizable characteristics that will describe snippet-based information linkages among relevant detected pathogens, allergens, antigens and antibodies that are shared by two or more samples of nucleic acids regardless of whether said samples are DNA or RNA snippets of pathogens, allergens, antigens or antibodies.

Having described preferred embodiments (which are intended to be illustrative and not limiting) for methods, processes and function, it is noted that modifications and variations can be made by persons skilled in the art in light of the above teachings. For example, fewer or additional steps may be provided while maintaining the functions and goals of the inventive method. The invention contemplates scalable operation whereby specific levels of irradiations, mixing of antiseptic air within the chamber and indoor may be accomplished by various techniques and processes intended to be covered by this Specification. While various embodiments may be constructed, the key is to balance the variables of disinfection to always provide treated air with at least 99.99% of pathogens eradicated. It is therefore to be understood that changes may be made in the particular embodiments of the invention disclosed which are within the scope and spirit of the invention.

1. A method for environmentally-modifying air within an indoor space containing disease-causing pathogens, comprising the steps of:
conveying a selected volume of air from the indoor space to a UVGI chamber;
irradiating the selected volume of air within the chamber while subjecting the selected volume of air to turbulence at a Reynold's Number (Re) between about 4,000 and about 5,000 to obtain treated air in less than 2 seconds having at least a log 4 reduction of disease-causing pathogens;
dispensing the treated air containing at least 99.99% inactivated pathogens from the chamber to permeate back in to the indoor space to dilute and diffuse the disease-causing pathogens remaining in the indoor space; and
inactivating disease-causing pathogens in situ during said irradiating step without contaminating or blurring the characteristics of the disease-causing pathogens so that the dispensed treated air consists of environmentally-modified breathable air with high acuity and sharpness of the inactivated pathogens.

2. The A method according to claim 1, further including the step of recirculating the diluted and diffused disease-causing pathogens through the UVGI chamber for homogenizing all air within the indoor space to at least a log 4 reduction of disease-causing pathogens separate and independent from an HVAC system servicing the indoor space.

3. The A method according to claim 1, wherein said dispensing step comprises dispensing the treated air from the chamber to permeate back in to the indoor space for diluting and diffusing the disease-causing pathogens remaining in the indoor space to reduce a viral load and a bacterial load of the disease-causing pathogens.

4. The method according to claim 1, wherein the irradiating step includes emitting ultraviolet radiation between 20-35 kWatts/m2 within the UVGI chamber.

5. The method according to claim 4, wherein said irradiating step consists of emitting ultraviolet radiation in the range of 250 and 280 nm, inclusive.

6. The method according to claim 5, wherein said irradiating step includes retaining the selected volume of air within the chamber for a dwell time of about a 1 second.

7. The method according to claim 6, wherein the irradiating step provide at least a log 5 reduction of disease-causing pathogens.

8. The method according to claim 7, wherein the inactivating step further includes preserving a biological characteristic of a disease-causing bacterial pathogen and preserving a genomic characteristic of a disease-causing viral pathogen so that the biological characteristics are present in the deactivated pathogens.

9. The method according to claim 8, wherein the preserved biological characteristics of the inactivated pathogens are adapted to safely trigger an immune response in an occupant of the indoor space without risk of infection.

10. The method according to claim 9, wherein the preserving step includes preserving the morphology, antigenic properties and immunogenic properties of disease-causing viral pathogens whereby the inactivated viral pathogens are adapted to induce production of virus-neutralizing antibodies in mammals present within the indoor space without risk of acquiring infectious disease.

11. The method according to claim 6, wherein the retaining step includes subjecting the selected volume of air to the Coanda Effect so that a portion of the selected volume of air hugs an interior surface of the UVGI chamber to increase dwell time.

12. The method according to claim 11, wherein said conveying and irradiating steps includes advancing the selected volume of air through passageways of varying widths to induce pressure and velocity differentials via the Bernoulli Principle to increase a throughput of treated air back in to the indoor space.

13. The method according to claim 12, wherein subjecting the selected volume of air to turbulence in combination with subjecting the selected volume of air to the Coanda Effect and advancing the selected volume of air through passageways of varying widths to induce pressure and velocity differentials provides sufficient irradiance dosage to dispense at least 100 cubic feet of treated air per minute with a log 6 reduction of disease-causing pathogens.

14. The method according to claim 13, wherein the dispensing step consists of dispensing the treated air containing at least 99.99999% deactivated pathogens from the chamber to permeate back in to the indoor space to dilute and diffuse the disease-causing pathogens remaining in the indoor space so that the entire indoor space contains antiseptic breathable air.

15. The method according to claim 6, wherein the irradiating and retaining steps provide effective levels of inactivating dwell time, wherein the conveying, dispensing and advancing steps provide throughput, and wherein the method further includes the step of balancing the effective levels of inactivating dwell time against throughput for obtaining a target irradiance as a product of flux, distance, time and UVGI wattage.

16. The method according to claim 1, wherein said irradiating step includes:
retaining the selected volume of air within the chamber for a dwell time of about a 1 second; and
advancing the selected volume of air through passageways of varying widths to induce pressure and velocity differentials via the Bernoulli Principle to increase a throughput of treated air back in to the indoor space.

17. The method according to claim 1, wherein said irradiating step includes:
subjecting the selected volume of air to the Coanda Effect so that a portion of the selected volume of air hugs an interior surface of the UVGI chamber to increase dwell time; and
advancing the selected volume of air through passageways of varying widths to induce pressure and velocity differentials provides sufficient irradiance dosage to dispense treated air per minute with a log 6 reduction of disease-causing pathogens.

18. The method according to claim 17, wherein the method further includes the step of balancing the effective levels of inactivating dwell time against throughput for obtaining a target irradiance as a product of flux, distance, time and UVGI wattage.

19. The method according to claim 17, wherein said irradiating step includes:
retaining the selected volume of air within the chamber for a dwell time of about a 1 second; and
emitting ultraviolet radiation between 20-35 kWatts/m2 within the UVGI chamber.

20. The method according to claim 19, wherein the method further includes the step of balancing the effective levels of inactivating dwell time against throughput for obtaining a target irradiance as a product of flux, distance, time and UVGI wattage.
